(19) United States
(12) Patent Application Publication
(10) Pub. No.: US 2022/0226620 A1
(43) Pub. Date: Jul. 21, 2022

(54) SYSTEMS AND METHODS FOR A BALLOON CATHETER SUPPORT SLEEVE

(71) Applicant: Sunnybrook Research Institute, Toronto (CA)

(72) Inventors: Bradley STRAUSS, Toronto (CA); Gilad CIBULSKI, Toronto (CA)

(73) Assignee: Sunnybrook Research Institute, Toronto, ON (CA)

(21) Appl. No.: 17/608,276

(22) PCT Filed: May 4, 2020
(86) PCT No.: PCT/CA2020/050594

§ 371 Date: Nov. 2, 2021

Related U.S. Application Data
(60) Provisional application No. 62/841,997, filed on May 2, 2019.

(51) Int. Cl. A61M 25/10

(52) U.S. Cl. CPC A61M 25/104; A61M 2025/1081

(57) ABSTRACT

A system and method for widening a narrowed blood vessel in a patient, the system including a guidewire, a balloon catheter having an enlarging balloon, and a tubular support sleeve having at least one support balloon. The support balloon is inflated to mechanically support the advancement of the balloon catheter into a narrowed segment of the blood vessel.

### CROSS-REFERENCE TO RELATED APPLICATIONS

The present application is based on and claims priority to U.S. Provisional Patent Application No. 62/841,997, filed May 2, 2019, and entitled “Balloon Catheter Support Sleeve,” which is hereby incorporated by reference herein in its entirety for all purposes.

### BACKGROUND

Within the human body, blood vessels can become narrowed. Arteries, for example, can become narrowed in discrete segments in disease states such as coronary artery disease. To widen narrowed blood vessels, angioplasty can be performed, in which a balloon catheter, sometimes having a stent coupled to the balloon for delivery, is fed through the circulatory system to the narrowed vessel and inflated. In some instances, however, the narrowed vessel is tortuous or calcified, making insertion of the balloon catheter into the narrowed vessel segment challenging, because the balloon catheter has little to no physical support to assist in urging it through the narrowed vessel.

Prior to inserting the balloon catheter, physicians will sometimes use a guideline extension device, which defines a hollow flexible tube, into the circulatory system. The guideline extension device can be fed through the vasculature of the patient until it is seated near the narrowed vessel segment. This device can provide support to a balloon catheter that is fed through the hollow channel of the guideline extension device. However, some guideline extension devices are very difficult to advance in certain types of coronary anatomy, cannot supply adequate support, back-out as the balloon catheter is attempted to advance, risk dissection when contrast injection is performed because the guideline extension device is inside the artery, and are typically very expensive.

### SUMMARY OF THE DISCLOSURE

It is an aspect of the present disclosure to provide a support sleeve for use with a balloon catheter. The support sleeve includes a sleeve portion, a support balloon, and an inflation tube. The sleeve portion is tubular in shape and has an internal diameter of a size and shape to receive a balloon catheter. The support balloon is coupled to an outer surface of the sleeve portion. The inflation tube is in fluid communication with the support balloon and a fluid source, the fluid source to inflate the support balloon, and the support sleeve being removably coupled to the balloon catheter.

In some embodiments, the support sleeve further includes a coupling portion that selectively couples the support sleeve to the balloon catheter.

In some embodiments, the coupling portion is a wire configured to contact the balloon catheter and a surface of the sleeve portion to restrict relative movement between the balloon catheter and the support sleeve.

In some embodiments, the coupling portion is a wire and a winch, the wire has a loop that is received around the balloon catheter, and the winch with the wire looped around the balloon catheter is configured to tighten or loosen the loop of the wire to selectively restrict or allow relative movement between the balloon catheter and the support sleeve.

In some embodiments, the support balloon is configured to be inflated to a diameter about equal to a diameter of a blood vessel adjacent to a narrowed blood vessel segment within a patient.

In some embodiments, the support balloon comprises: a first support balloon and a second support balloon, wherein the first support balloon is positioned to inflate outward from the outer surface of the sleeve portion and the second support balloon is positioned to inflate inward from an inner surface of the sleeve portion.

In some embodiments, the inflation tube is a hypo-tube

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 shows a support sleeve according to an embodiment.

FIG. 2 shows a system for enlarging a narrow passage within the body of a patient according to an embodiment.

FIG. 3 shows the system of FIG. 2 having an inflated support balloon.

FIG. 4A shows a partial view of a support sleeve according to an embodiment.

FIG. 4B shows a cross section through the partial view of the support sleeve shown in FIG. 4A.

FIG. 5 shows a cross-sectional side view of a system for enlarging a blood vessel.

FIG. 6 shows a cross-sectional side view of another system for enlarging a blood vessel.

FIG. 7 shows a flow chart of a process for widening a narrowed blood vessel according to an embodiment.

### DETAILED DESCRIPTION

Described here are systems and methods for widening a narrowed blood vessel within a patient. Embodiments described in the present disclosure facilitate angioplasty balloon catheter and stent catheter advancement, which in some instances may be after the lesion has been crossed with a guidewire, in arteries that are tortuous and/or calcified. In many cases, advancing balloon catheters can be challenging due to inadequate guider support. When the balloon catheter meets resistance, the guiding catheter can be pushed out of the coronary artery ostium and the balloon catheter cannot progress.

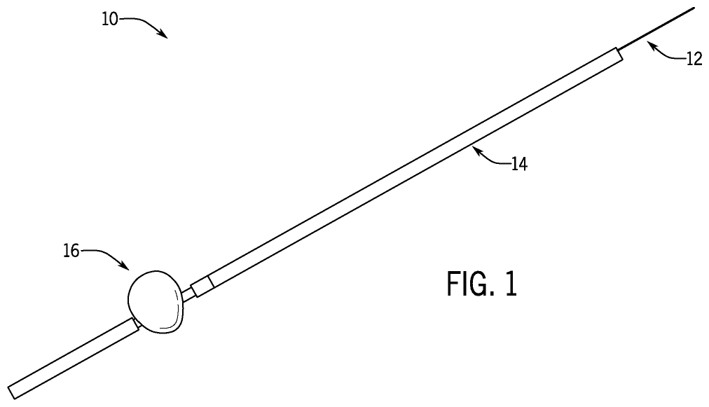

Referring first to FIG. 1, a support sleeve 10 according to an embodiment is illustrated. The support sleeve 10 includes an inflation tube 12, a tubular sleeve portion 14, and a support balloon 16. In general, the tubular sleeve portion 14 is sized to receive a catheter, such as a balloon catheter. The support balloon 16 is coupled to the outer surface of the tubular sleeve portion 14 such that when inflated the support balloon extends away from the outer surface of the tubular sleeve portion 14 to make contact with the inner surface of a vessel lumen. The inflation tube 12 and the support balloon 16 are in fluid communication with each other so that a syringe can be coupled to the inflation tube 12 and air, liquid, or another fluid can be injected through the inflation tube 12 into the support balloon 16 in order to expand the support balloon 16. The inflation tube 12 can be a hypo-tube or any small tube having a diameter that allows the inflation tube 12 to be fed through the vascular system of a patient.

The support balloon 16 can be composed of a compliant material such that the support balloon 16 can reach its fully-inflated diameter with very little pressure applied by the syringe. In one non-limiting example, in the fully-inflated state, the support balloon 16 expands to a spherical shape having a diameter about equal to the diameter of the artery where the support sleeve 10 will placed during a blood vessel widening procedure. The extent of the inflation of support balloon 16 will depend on the fluid pressure applied via the syringe. In other configurations, the support balloon 16 can have different shapes or geometries. After inflation, the support balloon 16 firmly holds the support sleeve 10 in place within the blood vessel and can act as structure against which the balloon catheter 24 can be advanced. In some embodiments, and as illustrated in FIG. 1, the support balloon 16 extends around the entire circumference of the sleeve portion 14, however in alternative configurations, the balloon 16 can extend along a portion of the circumference of the sleeve portion 14 (e.g., an arc length of the circumference of the sleeve portion 14).

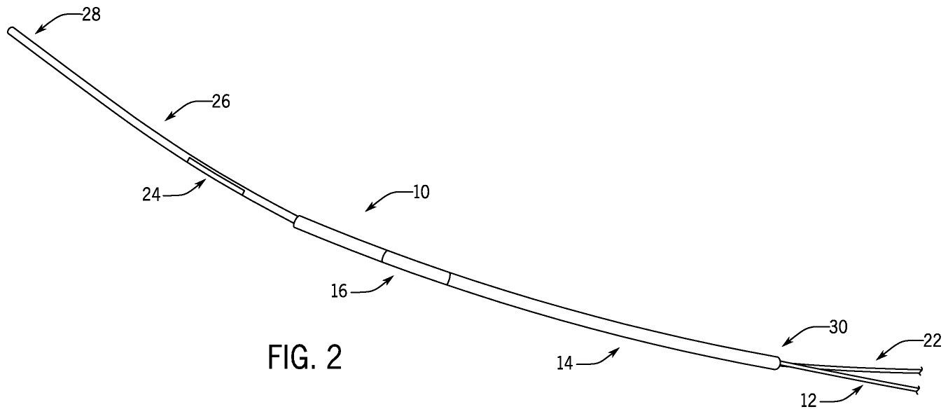

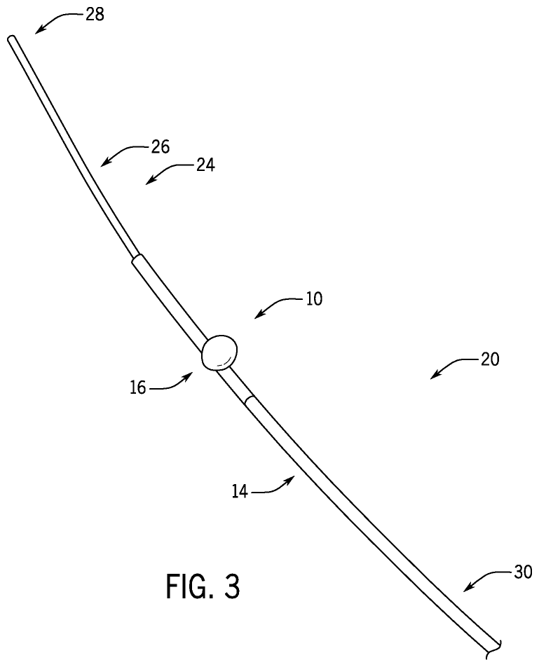

In some forms, the support sleeve 10 can include multiple support balloons 16. In these instances, some support balloons 16 can be positioned to expand from the outer surface of the tubular sleeve portion 14 to contact and press against the inner wall of a patient's blood vessel. This configuration prevents the support sleeve 10 from backing away from the narrowed blood vessel segment. Some support balloons 16 can be positioned to expand inward from the inner surface of the tubular sleeve portion 14 to contact a guidewire 22 (FIGS. 2-3) extending through the tubular sleeve portion 14. This configuration helps to prevent backing out of the balloon catheter 24 during and after advancement of the balloon catheter 24 into the narrowed blood vessel segment. The support sleeve 10 can have any combination of support balloon 16 configurations, such as two support balloons expanding outward, two support balloons expanding inward, one support balloon expanding inward and one expanding outward, and so on.

Referring next to FIGS. 2 and 3, a system 20 for enlarging a narrow passage within the body of a patient is shown. The system 20 includes a guidewire 22, a balloon catheter 24 having an enlarging balloon 26, and the support sleeve 10. The balloon catheter 24 is advanced over the guide wire 22, and the support sleeve 10 has an internal diameter of a size and shape to receive the balloon catheter 24 in its deflated state. In use, the balloon catheter 24 and the guide wire 22 extend through the tubular sleeve portion 14. Thus, when the support sleeve 10 is placed for vessel widening, the inflation tube 12 and the guide wire 22 extend in parallel back through the circulatory system to the entrance of system 20 into the body.

In some forms, the support sleeve 10 includes a coupling portion and/or the balloon catheter 24 includes a coupling portion. The coupling portions can include an adhesive-based coupling, a mechanical coupling, or any other coupling arrangement that provides selective coupling of the support sleeve 10 to the balloon catheter 24. For example, the mechanical coupling can be additional balloons or a double walled balloon, the expansion of which can hold the support sleeve 10 and the balloon catheter 24 together frictionally. Further, the mechanical coupling can include other frictional mechanisms, scaffolding, or a hook and loop structure that provide selective coupling and decoupling. Balloon catheter 24 has a distal end 28 and a proximal end 30, the distal end 28 being the leading tip of the balloon catheter 24 and the proximal end 30 being located adjacent to a guidewire export port of the balloon catheter 24.

The coupling portions described above selectively couple the support sleeve 10 and the balloon catheter 24 such that the support sleeve 10 is positioned proximal to the enlarging balloon 26 of balloon catheter 24, but distal to the guidewire export port. In some other instances, the support sleeve 10 can be coupled at the distal end of the balloon catheter 24, such that the support sleeve 10 is the leading edge of the combined device that is introduced into the patient's vasculature. The support sleeve 10 can be selectively coupled and decoupled from the balloon catheter 24 so that the support sleeve 10 and balloon catheter 24 can be advanced together through the vasculature of the patient, but the balloon catheter 24 can also be advanced separately from the support sleeve 10 once the system 20 reaches the narrowed blood vessel segment.

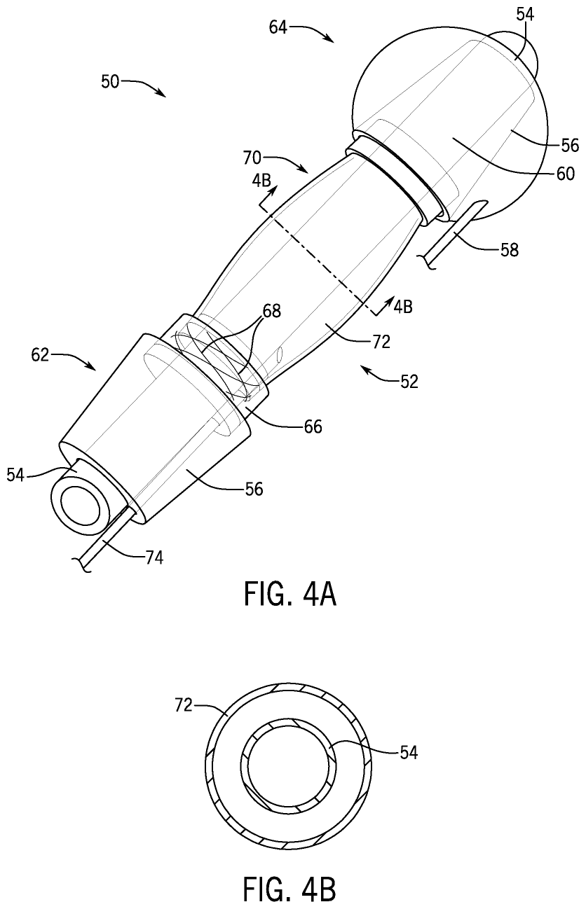

FIG. 4A shows a top view of another system 50 for enlarging a passage within the body of a patient, which is a specific implementation of the system 20. Thus, the previous description of the system 20 also pertains to the system 50. The system 50 also includes a support sleeve 52, and a balloon catheter 54. As shown in FIG. 4A, the support sleeve 52 has a region with layers that have been removed to expose more internal layers for visual clarity. Thus, layers (or components) of the support sleeve are generally intended to be coaxially disposed relative to each other and to extend along an axis (axial direction) together, as shown in FIG. 4B. In other words, the region with the layers removed in FIG. 4A is not intended to be the actual structure of the support sleeve 52, and rather is shown for clearer illustration of the internal components of the support sleeve 52.

Similarly to the support sleeve 10, the support sleeve 52 also includes a tubular sleeve portion 56, and an inflation tube 58 in fluid communication with a support balloon 60. The tubular sleeve portion 56 is sized (or otherwise dimensioned) to be inserted inside any number of vascular structures in the patient (e.g., veins, arteries, etc.). The tubular sleeve portion 56 has a proximal end 62, an opposite distal end 64, and a bore therethrough (e.g., extending along the axial direction). As shown, the distal end 64 of the tubular sleeve portion 52 has an arcuate (or tapered) shape as the distal end 64 of the tubular sleeve portion 56 extends farther distally. In some embodiments, the cross-sectional area of the distal end 64 can (gradually) decrease at the distal end 64 of the tubular sleeve portion 56 extends farther distally. This gradual decreasing in cross-sectional area may more easily allow the tubular sleeve portion 56 to be traversed through the vascular structure of the patient. The support balloon 60 is coupled to an exterior surface of the distal end 64 of the tubular sleeve portion 56, and can be selectively inflated to firmly hold and support the support sleeve 52 at a particular location within the vasculature of the patient. Similarly to the support balloon 16, the support balloon 60 can extend around the entire circumference of a portion of the exterior surface of the tubular sleeve portion 56. However, in alterative embodiments, the support balloon 60 can extend along only a portion of the circumference of the exterior surface of the tubular sleeve portion 56 (e.g., from and to opposing ends of the circumference, such as 180°). In other cases, the support balloon can have two independently inflatable portions positioned on opposing sides of the tubular sleeve portion and interfaced with respective inflation tubes.

The support sleeve 52 also includes a liner 66 that is coupled to the interior surface of the tubular sleeve portion 56. The liner 66 can be relatively thin and formed out of a flexible material (e.g., polytetrafluoroethylene (“PTFE”)). As shown in FIG. 4A, the liner 66 includes a braided filament 68 that provides flexibility and structurally reinforces the liner 66. The braided filament 68 can have individual filaments of a particular size and can be formed out of various materials (e.g., metals, plastics, etc.). The braided filament 68 is illustrated as having four alternating filaments in a helical pattern that is roughly equidistant to adjacent filaments, however in alternative configurations, other numbers of filaments or different styles (or types) of the braiding pattern can be used for the braided filament 68. In some cases, the liner 66 can be sandwiched between the braided filament 68, while in other cases, the braided filament 68 can be coupled to a specific surface (e.g., the interior or exterior surface) of the liner 66. In alternative configurations, the liner 66 can be removed and the braided filament can be coupled to the interior surface of the tubular sleeve portion 56. Thus, generally, the braided filament 68 is structured as having a tube shape, however the overall shape of the braided filament 68 can be adjusted accordingly, based on, for example, the desired flexibility of the support sleeve 52 (e.g., the braided filament can embody different shapes, such as a rectangular prism, and octagonal prim, etc.).

The support sleeve 52 also includes a coupling portion 70 that selectively allows or restricts advancement of the balloon catheter 54 with or without the support sleeve 52. In other words, the coupling portion 70 allows the balloon catheter 54 to be removably coupled to the support sleeve 52, such that when the balloon catheter 54 is advanced into the vasculature of the patient, the balloon catheter 54 can be advanced with the support sleeve 52 (when coupled), or alternatively, advanced alone (when the balloon catheter 54 is decoupled from the support sleeve 52). In the illustrated embodiment of FIGS. 4A and 4B, the coupling portion 70 is implemented as being a balloon 72 in fluid communication with an inflation tube 74. The balloon 72 is coupled to the liner 66 (e.g., the interior surface) and allows the support sleeve 52 to be removably coupled to the balloon catheter 54. For example, when the balloon 72 is inflated, a surface of the balloon 72 contacts the balloon catheter 54 so as to couple the support sleeve 52 to the balloon catheter 54. This way, the support sleeve 52 and the balloon catheter 54 are advanced together along the vasculature of the patient. In other words, relative movement between the support sleeve 52 and the balloon catheter 54 is restricted. Alternatively, when the balloon 72 is deflated (e.g., when reaching a narrowed or calcified vessel), the surface of the balloon 72 is retracted (entirely or somewhat) away from the balloon catheter 54, and thus allowing the balloon catheter 54 to freely move away from the support sleeve 52. In other words, relative movement between the balloon catheter 54 and the support sleeve 52 is allowed. In some cases, the balloon 72 (or other coupling portion 70, such as those described below) can adjust the force (or effort) required by the practitioner to advance the balloon catheter 54 relative to the support sleeve 52. For example, different deflation (or inflation) levels (e.g., pressure, fluid volume, etc.) can adjust the amount of friction between the balloon 72 and the balloon catheter 54, which impacts how easily or hard (e.g., the force) required to translate the balloon catheter 54 relative to the balloon 72. In other words, the balloon 72 (or other coupling portion 70) can adjust a degree of coupling between the balloon 72 (or generally the support sleeve 52) and the balloon catheter 54.

Although the inflation tube 58 is generally coupled to and extends along the tubular sleeve portion 56. However, in alternative embodiments, the inflation tube 58 can be directed into the tubular sleeve portion 56 (or additionally other layers, such as the liner 66) to extend along different components to reach the proximal end of the support sleeve 52 (e.g., extend along the internal surface of the tubular sleeve portion 56, extend along the interior surface of the liner 66, etc.).

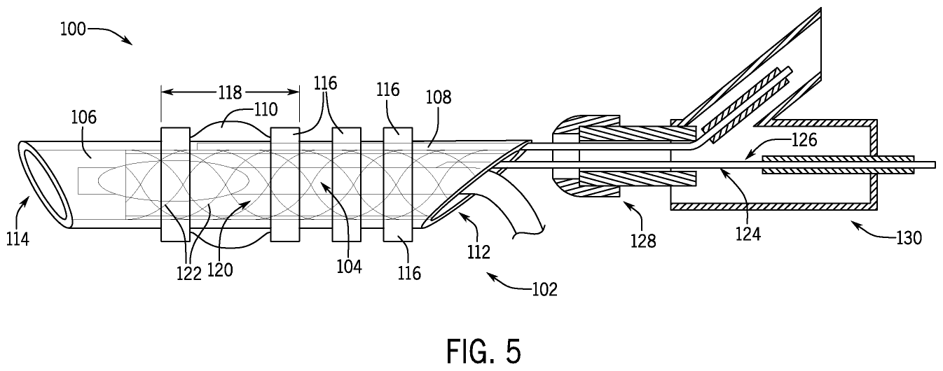

FIG. 5 shows a cross-sectional side view of another system 100 for enlarging a passage within the body of a patient, which is similar to the previously described systems 20, 50. Thus, the previous description of the systems 20, 50 also pertain to the system 100. The system 100 also includes a support sleeve 102, and a balloon catheter 104. The support sleeve 102 includes a tubular sleeve portion 106, and an inflation tube 108 in fluid communication with a support balloon 110. The tubular sleeve portion 106 is sized to be inserted inside any number of vascular structures in the patient. In some embodiments, the tubular sleeve portion 106 is a hypo-tube. The tubular sleeve portion 56 has a proximal end 112, an opposite distal end 114, and a bore therethrough. As shown, the support balloon 110 is coupled to an exterior surface of the tubular sleeve portion 106 and is positioned between the ends 112, 114 of the tubular sleeve portion 56. In particular, the tubular sleeve portion has protrusions 116 that extend radially outward from the exterior surface of the tubular sleeve portion 106, and the support balloon 110 can be positioned between adjacent protrusions 116. In some cases, the protrusions 116 can be formed of a material that has a lower sliding coefficient of friction (and static coefficient of friction) than the tubular sleeve portion 106, which can allow the support sleeve 102 to be easily slide across the vascular structure being traversed (e.g., via the protrusions 116). In some embodiments, a given protrusion 116 can extend around the entire circumference (or portions of) the circumference of the tubular sleeve portion 106. Additionally, adjacent protrusions 116 can be separated from each other by the same distances, or the separation distances can vary (e.g., along the axial direction of the tubular sleeve portion). The protrusions 116 are illustrated as being substantially flat (e.g., having a plateau), although in alternative embodiments, other shapes can be utilized (e.g., hemispheres), based on the desired degree of slidably.

As described above, sets of adjacent protrusions 116 can receive a support balloon (e.g., the support balloon 110). This can be advantageous in that the practitioner can specifically tailor the anchoring ability by selecting the number or size of support balloons 110, based on the patient's anatomy, the type of vascular structure, the anatomical location, etc. The support balloon(s) 110 can be selectively inflated to firmly hold (or otherwise anchor) the support sleeve 102 at a particular location within the vasculature of the patient. In some embodiments, a given balloon 110 and the adjacent protrusions 116 can have various axial lengths 118. In some specific configurations, the axial length 118 can be less than 20 mm, less than 10 mm, in a range between 5 mm and 15 mm, etc. In some embodiments, the protrusions 116 can extend away from the exterior surface of the tubular sleeve portion 106 by a height that does not significantly increase the outer diameter of the support sleeve 102. As shown, the support balloon 110 in an inflated state has a greater height than the height of the protrusions 116, and which can be in a range of 5 mm to 25 mm. In some cases, the height difference between the inflated balloon 110 and the protrusions 116 can be about 1 mm (e.g., the height of the inflated balloon 110 being 5 mm). In some embodiments, the axial length of the protrusions 116 can be less than or equal to 20 mm. In some embodiments, the balloon(s) 110 can be formed out of various materials, such as, for example, polyurethane, Pebax®, silicon, etc.

As shown, the support sleeve 102 also includes a liner 120 having a thickness, and which is coupled to the interior surface of the tubular sleeve portion 106. The liner 120 can be relatively thin and formed out of a flexible material (e.g., polytetrafluoroethylene (“PTFE”)). As also shown in FIG. 5, the liner 120 includes a braided filament 122 that provides flexibility and structurally reinforces the liner 120. The braided filament 122 can have individual filaments of a particular size and can be formed out of various materials (e.g., metals, plastics, etc.). The braided filament 122 is illustrated as having a crossed-hatched pattern, however in alternative configurations, other numbers of filaments or different styles (or types) of the braiding pattern can be used for the braided filament 122. In some cases, the liner 120 can be sandwiched between the braided filament 122, while in other cases, the braided filament 122 can be coupled to a specific surface (e.g., the interior or exterior surface) of the liner 120. In alternative configurations, the liner 120 can be removed and the braided filament can be coupled to the interior surface of the tubular sleeve portion 106. Thus, generally, the braided filament 122 is structured as having a tube shape, however the overall shape of the braided filament 122 can be adjusted accordingly, based on, for example, the desired flexibility of the support sleeve 102 (e.g., the braided filament can embody different shapes, such as a rectangular prism, and octagonal prim, etc.). As also shown, the liner 120 (and the braided filament 122) only extend along a portion of the tubular sleeve portion 106. In some specific examples, the linear (and the braided filament 122) have an axial length in a range between 30 mm and 120 mm.

The support sleeve 102 also includes a coupling portion 124 that selectively allows or restricts advancement of the balloon catheter 104 with or without the support sleeve 102. In other words, the coupling portion 124 allows the balloon catheter 104 to be removably coupled to the support sleeve 102, such that when the balloon catheter 104 is advanced into the vasculature of the patient, the balloon catheter 104 can be advanced with the support sleeve 102 (when coupled), or alternatively, advanced alone (when the balloon catheter 104 is decoupled from the support sleeve 102). In the illustrated embodiment of FIG. 5, the coupling portion 124 is implemented as being a fixation wire 126. The fixation wire 126 can be structured as being a typical wire formed of metal (e.g., a stainless steel wire) and being thread-like, and in other cases, the fixation wire 126 can embody various other forms, shapes, etc. Thus, the fixation wire 126 need not be only long and thin, and rather the fixation wire 126 can be plate-like, etc. In some embodiments, a portion (or all of) the fixation wire 126 can be encapsulated (or disposed on a surface) with a coating layer that can have a higher sliding (and static) coefficient of friction than the fixation wire 126 itself. This coating can thus provide varying levels of resistance to movement of the balloon catheter 104 relative to the fixation wire 126. In some embodiments, the coating can also prevent edges (or ends) of the fixation wire 126 from undesirably puncturing components of the system 100.

The fixation wire 126 generally allows the support sleeve 102 to be removably coupled to the balloon catheter 104. For example, when the fixation wire 126 is inserted into the tubular sleeve portion 106, the fixation wire 126 contacts (or is wedged between) the balloon catheter 104 and the liner 120 (or the braided filament 122) to temporarily couple the balloon catheter 104 to the liner 120 of the support sleeve 102 (e.g., via the fixation wire 126). The contact of the fixation wire 126 allows the balloon catheter 104 and the support sleeve 102 to be advanced together along the vasculature of the patient. Stated another way, relative movement between the balloon catheter 104 and the support sleeve 102 is prevented. Alternatively, when the fixation wire 126 is removed (e.g., pulled out of contact with the balloon catheter 104 and the liner 120) the balloon catheter 104 is free to move (or translate) away from the support sleeve 102. Thus, relative movement between the balloon catheter 104 and the support sleeve 102 is allowed. Similarly to the balloon 72 above, the fixation wire 126 can be advanced (or retreated) to increase (or decrease) the force required to advance the balloon catheter 104 relative to the support sleeve 102. For example, as the fixation wire 126 is advanced farther relative to the proximal end of the support sleeve 102, more surface area of the fixation wire 126 contacts the balloon catheter 104 (and the liner 120), and thus increases the force required to create relative movement between the fixation wire 126 and the balloon catheter 104. Similarly, as the fixation wire 126 is retreated closer to the proximal end of the support sleeve 102, less surface area of the fixation wire 126 contacts the balloon catheter 104 (and the liner 120), and thus decreases the force required to create relative movement between the fixation wire 126 and the balloon catheter 104.

As shown, the inflation tube 108 is generally coupled to and extends along the tubular sleeve portion 106 (e.g., to reach the balloon 110). However, in alternative embodiments, the inflation tube 108 can be directed into the tubular sleeve portion 106 (or additionally other layers, such as the liner 120) to extend from the balloon 110 and along different components to reach the proximal end of the support sleeve 102 (e.g., extending along the internal surface of the tubular sleeve portion 106, extending along the interior surface of the liner 120, etc.). In some embodiments, the inflation tube 108 can be formed out of polyimide.

In some embodiments, and as illustrated, the support sleeve 102 also includes a port adapter 128, and a dual port attachment 130. The port adapter 128 can be coupled to the tubular sleeve portion 106, although in the illustrated embodiment of FIG. 5 the port adapter 128 is separated from the tubular sleeve portion 106. As shown, a proximal end of the port attachment 128 is coupled (e.g., by threaded engagement, adhesive, etc.) to a first end of the dual port attachment 130, while the distal end of the port attachment 128 is tapered (e.g., along an axial direction towards the distal end). The opposing second end of the dual port attachment 130 includes two distinct ports that eventually converge to the single bore defined by the port adapter 128. In some specific implementations, the dual port attachment 130 is a y-luer. As illustrated in FIG. 5, the two port configuration allows one port to independently receive the fixation wire 124, while the second port (e.g., coaxially positioned to the bore of the port adapter 128) independently receives the inflation tube 108 (e.g., to eventually connect to a fluid source). In this case, as described above, the fixation wire 124 can be more easily manipulated in its own independent port. Additionally, as in the illustrated embodiment, the inflation tube 108 is coupled to the port adapter 128.

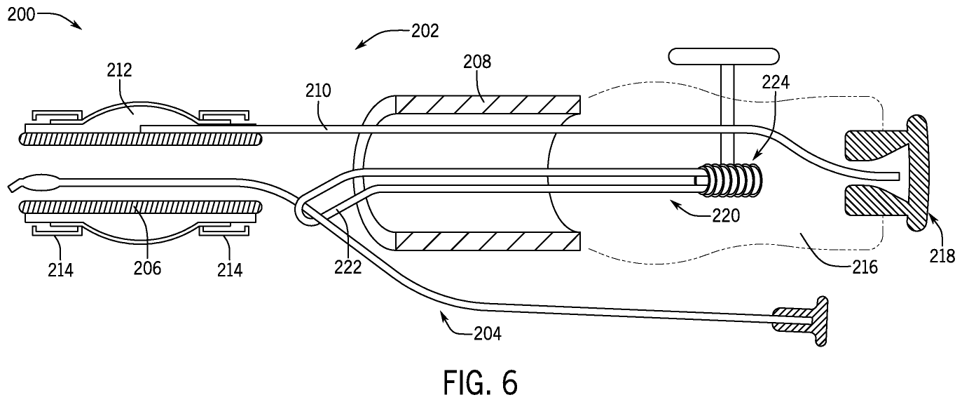

FIG. 6 shows a cross-sectional side view of another system 200 for enlarging a passage within the body of a patient, which is similar to the previously described systems 20, 50, and 100. Thus, the previous description of the systems 20, 50, 100 also pertain to the system 200. The system 200 also includes a support sleeve 202, and a balloon catheter 204. The support sleeve 202 includes a first tubular sleeve portion 206, a second tubular sleeve portion 208, and an inflation tube 210 in fluid communication with a support balloon 212. The first tubular portion 206 is similarly structured to the tubular portion 106. For example, the first tubular portion 206 also includes protrusions 214, and the support balloon 212 being coupled to the exterior surface of the first tubular portion 206 between adjacent protrusions 214. As also previously described, the first tubular portion 206 can also include a liner with a braided filament.

The second tubular portion 208 is illustrated as having a bore directed therethrough. In some specific embodiments the second tubular portion 208 is a hypo-tube. In some embodiments, a portion of the second tubular portion 208 can be coupled to the first tubular portion 206. In this case, the first and second tubular portions 206, 208 would still be separated by a distance (or aperture) to receive the balloon catheter 204. As shown, the inflation tube 210 is coupled to and extends along the exterior surface of the first tubular portion 206, extends through (and can be coupled to an interior surface of) the second tubular portion 208, extends into (or externally relative to) a handle 216 of the support sleeve 102, and is inserted into a port adapter 218 of the support sleeve 102. The port adapter 218 is configured to receive a fluid infusion device (e.g., a syringe), and once the fluid infusion device is interfaced with the port adapter 218, the fluid infusion device is also in fluid communication with the inflation tube 210. This way, the fluid infusion device can provide a fluid to the support balloon 212 via the inflation tube 210.

As shown, the support sleeve 202 also includes a coupling portion 220 that selectively allows or restricts advancement of the balloon catheter 204 with or without the support sleeve 202. In other words, the coupling portion 220 allows the balloon catheter 204 to be removably coupled to the support sleeve 202, such that when the balloon catheter 204 is advanced into the vasculature of the patient, the balloon catheter 204 can be advanced with the support sleeve 202 (when coupled), or alternatively, advanced alone (when the balloon catheter 204 is decoupled from the support sleeve 202). In the illustrated embodiment of FIG. 6, the coupling portion 220 is implemented as being a wire 222 (or other tethering structure) interfaced with a winch 224. The winch 224 is illustrated as having a rotatable handle that interfaces with gears, other rotating components such as a shaft, etc., to draw in (or out, such as to provide slack) one end of the wire 222, while the other end of the wire 222 can be fixed to a portion of the winch 224 (e.g., the rotatable shaft of the winch) or other structure. The wire 222 can have an appropriate size, and can be made out of various materials. For example, the wire 222 can be formed out of a super elastic metal (e.g., nitinol), and can be made to have a thickness being, for example, 0.004 inches, 0.0006 inches, etc. In some cases, the wire 222 can be rounded (e.g., free of edges), and in other cases the wire 222 can be formed of other materials, such as plastics.

The wire 222 (and the winch 224) generally allows the support sleeve 202 to be removably coupled to the balloon catheter 204. For example, as shown the wire 222 is looped around the balloon catheter 204, and thus when the wire 222 is taught (e.g., via tightening by the winch 224), the loop of the wire 222 becomes smaller and increases the contact between the wire 222 and the balloon catheter 204. As such, this tightening of the wire 222 temporarily couples the balloon catheter 204 to the wire 222. This restriction by the wire 222 allows the balloon catheter 204 and the support sleeve 202 to be advanced together along the vasculature of the patient. Stated another way, relative movement between the balloon catheter 204 and the support sleeve 202 is prevented. Alternatively, when the wire 222 is loosened (e.g., via rotating the winch 224 in the opposing direction), the loop of the wire 222 increases in size and decreases the contact between the wire 222 and the balloon catheter 204. As such, this loosing of the wire 222 temporarily decouples the balloon catheter 204 from the wire. This loosening allows the balloon catheter 204 to freely translate relative to the support sleeve 202. Similarly, to the other systems described above, a degree of tightness (or looseness) of the loop of the wire 222 can increase (or decrease) the force required to advance the balloon catheter 204.

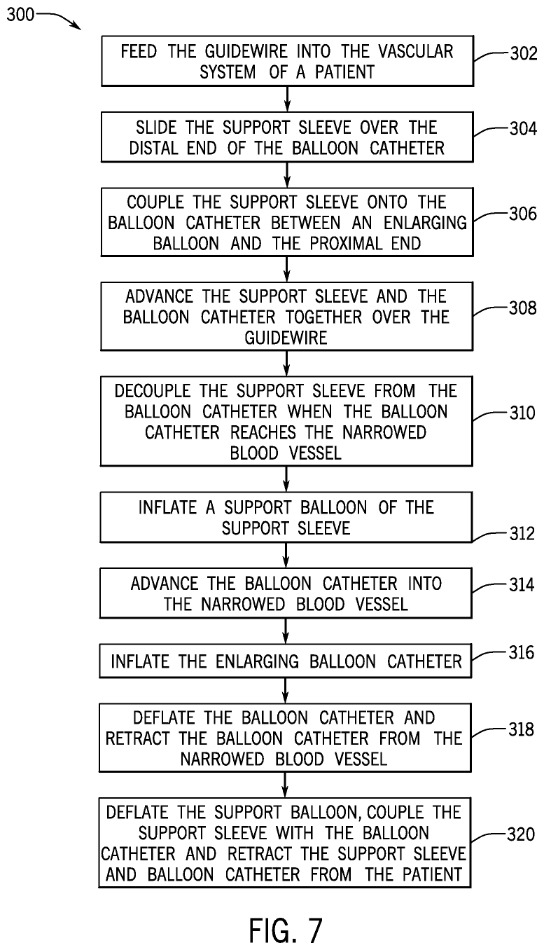

FIG. 7 shows a flow chart of a process 300 for widening a narrowed blood vessel. In some embodiments, the process 300 can utilize a guidewire, a balloon catheter having a distal end and a proximal end, a support sleeve having a support balloon, and other components. At 302, process 300 includes placing and feeding a guidewire and a balloon catheter into the vascular system of the patient. For example, a physician can feed and place the guidewire, followed by feeding and placing the balloon catheter up to the narrowed blood vessel segment. If the balloon catheter can be advanced into place to widen the narrowed segment, then the physician can do so and complete the procedure. If the balloon catheter cannot be advanced through the narrowed segment, the balloon catheter can be retracted back out of the patient. In some forms, the physician does not need to test for a problem with the advancement of the balloon catheter, but rather can begin the procedure with the support sleeve attached to the balloon catheter.

At 304, process 300 includes sliding the support sleeve over the balloon catheter. For example, the support sleeve can be slid over the distal end of the balloon catheter and advanced to a desired position. At 306, process 300 includes temporarily coupling the balloon catheter to the support sleeve. For example, the balloon can be temporarily coupled to the balloon catheter between an enlarging balloon of the balloon catheter and the proximal end.

At 308, process 300 includes advancing the support sleeve and the balloon catheter together over the guidewire. For example, once the balloon catheter is temporarily coupled to the support sleeve, the balloon catheter and the support sleeve can be advanced together through the patient's circulatory system until the narrowed vessel segment is reached.

At 310, process 300 includes decoupling the support sleeve from the balloon catheter when the balloon catheter reaches the narrowed blood vessels. In some cases, the practitioner can visually determine (e.g., on a medical image of the procedure), or can determine in a tactile sense (e.g., from resistance, or difficulty in advancement of balloon catheter with the support sleeve) that the balloon catheter has reached the narrowed blood vessel. Regardless, the balloon catheter can be decoupled from the support sleeve to allow the balloon catheter to translate (or move) relative to the support sleeve.

At 312, process 300 includes inflating the balloon of the support sleeve. After the balloon catheter is decoupled from the support sleeve, the support balloon can be inflated to contact and thus anchor the support sleeve at a location within the blood vessel.

At 314, process 300 includes advancing the balloon catheter into the narrowed blood vessel. For example, after the support sleeve is properly positioned and secured (e.g., anchored by the inflation of the support balloon), the balloon catheter can be advanced into the narrowed blood vessel segment and positioned accordingly. For example, the enlarging balloon of the balloon catheter can be positioned entirely within the narrowed portion of the vessel desired to be expanded. In some cases, use of the support sleeve may allow the balloon catheter to be advanced beyond a first narrowed portion of the patient's vasculature, but a second narrowed portion may be encountered before the balloon catheter can reach the desired segment. In these instances, the balloon of the support sleeve can be deflated and the support sleeve advanced over the balloon catheter at the second narrowed portion. The support sleeve can then be redeployed by inflating the balloon of the support sleeve at this second, more distal narrowed portion of the patient's vasculature. This process can be repeated several times, if necessary, to reach the desired segment in the patient's vasculature.

At 316, process 300 include inflating the balloon catheter. For example, after the balloon catheter has been successfully advanced (and positioned accordingly), the balloon catheter is inflated to widen the narrowed segment of the vessel to be expanded. If needed, a stent can also be placed by the balloon catheter as the enlarging balloon expands.

At 318, process 300 can include deflating the balloon catheter and retracting the balloon catheter from the narrowed (and now expanded) blood vessel. For example, when sufficient enlarging balloon inflation and vessel widening has occurred or if for any other reasons the balloon catheter needs to be removed from the patient, the enlarging balloon can be deflated and the balloon catheter can be retracted.

At 320, process 300 can include deflating the support balloon, coupling (temporarily) the support sleeve to the balloon catheter, and retracting the balloon catheter with the support sleeve from the patient. For example, once the balloon catheter is retracted to an appropriate position, the balloon catheter can be coupled to the support sleeve (with the support balloon inflated). Then, the support balloon can be deflated and the balloon catheter and support sleeve can be retracted together from the patient. Although this description has set out specific embodiments of a method of widening a narrowed blood vessel, it should be noted that a number of these steps may take place in a different order than described.

The present disclosure has described one or more preferred embodiments, and it should be appreciated that many equivalents, alternatives, variations, and modifications, aside from those expressly stated, are possible and within the scope of the invention.

1. A support sleeve for use with a balloon catheter, the support sleeve comprising:
a sleeve portion, tubular in shape, and having an internal diameter of a size and shape to receive a balloon catheter;
a support balloon coupled to an exterior surface of the sleeve portion; and
an inflation tube in fluid communication with the support balloon, wherein the inflation tube enables a fluid to be provided to the support balloon in order to inflate the support balloon.

2. The support sleeve of claim 1, further comprising a coupling portion that provides for selectively coupling the support sleeve to a balloon catheter arranged within the internal diameter of the sleeve portion.

3. The support sleeve of claim 2, wherein the coupling portion is a wire configured to contact the balloon catheter and an internal surface of the sleeve portion to restrict relative movement between the balloon catheter and the support sleeve.

4. The support sleeve of claim 3, wherein the wire is a flat-shaped wire having a width that is greater than a height.

5. The support sleeve of claim 3, wherein the wire is coated with a coating layer having a coefficient of friction sufficient to restrict the relative movement between the balloon catheter and the support sleeve.

6. The support sleeve of claim 2, wherein the coupling portion is a wire and a winch, the wire has a loop for receiving the balloon catheter, and
wherein the winch is operable to tighten or loosen the loop of the wire to selectively restrict or allow relative movement between the balloon catheter and the support sleeve.

7. The support sleeve of claim 4, further comprising:
a handle proximal to the support sleeve, wherein the winch is coupled to the handle; and
a hypo-tube extending from the handle to a position adjacent a proximal end of the support sleeve, wherein the wire and the inflation tube are arranged within the hypo-tube.

8. The support sleeve of claim 1, wherein the support balloon is configured to be inflated to a diameter about equal to a diameter of a blood vessel adjacent to a narrowed blood vessel segment within a patient.

9. The support sleeve of claim 1, wherein the support balloon comprises:
a first support balloon and a second support balloon, wherein the first support balloon is positioned to inflate outward from the outer surface of the sleeve portion and the second support balloon is positioned to inflate inward from an inner surface of the sleeve portion.

10. The support sleeve of claim 1, wherein the inflation tube is a hypo-tube.

11. The support sleeve of claim 1, wherein the support sleeve comprises a flexible tube having a braided filament coupled to an interior surface of the flexible tube.

12. The support sleeve of claim 1, wherein the sleeve portion has a length between 30 mm and 120 mm.

13. The support sleeve of claim 12, wherein the support balloon has an axial length between 5 mm and 20 mm.
